(19) United States
(12) Patent Application Publication
(10) Pub. No.: US 2008/0262187 A1
(43) Pub. Date: Oct. 23, 2008

(54) Blends of Diamines Having Reduced Color

(75) Inventors: John Y. Lee, Baton Rouge, LA (US); David W. Owens, Baton Rouge, LA (US); Paul L. Wiggins, Baton Rouge, LA (US); Richard D. Glass, Baton Rouge, LA (US); Michael J. Wilhelm, Baton Rouge, LA (US); Robert Phillip Slicker, Baton Rouge, LA (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 12/096,128

(22) PCT Filed: Dec. 21, 2006
(86) PCT No.: PCT/US2006/062493

§ 371 Date: Jun. 4, 2008

Related U.S. Application Data
(60) Provisional application No. 60/755,550, filed on Dec. 30, 2005.

(51) Int. Cl. C08G 71/02; C07C 211/54; C08G 71/04; C07C 211/50

(52) U.S. Cl. 528/68; 564/330; 564/443; 252/183.11

(57) ABSTRACT

This invention provides a blend which comprises (i) at least one aromatic primary diamine, with which has been blended a color-minimizing amount of at least one N,N-dmydrocarbylhydroxylamine, wherein the aromatic primary diamine is in the form of one benzene ring having two primary amino groups on the ling, which amino groups are meta or para relative to each other, and in which each position ortho to a primary amino group bears an alkyl group, and (ii) at least one aromatic secondary diamine having a Gardner color number no more than about 7, wherein said aromatic secondary diamine either is in the form of one benzene ring having two secondary amino groups on the ring, or is in the form of two benzene rings connected by an alkylene bridge and having one secondary amino group on each ring. Optionally, at least one N,N-dihydrocarbylhydroxylamine has been blended with the aromatic secondary diamine.

### TECHNICAL FIELD

This invention relates to blends of aromatic diamines, which aromatic diamines have reduced color.

### BACKGROUND

Aromatic diamines are indicated to be useful as chain extenders in the preparation of polyurethane, polyurea, and polyurethane-urea polymers and/or as curing agents for epoxy resins. Blends of aromatic primary diamines and aromatic secondary diamines are known. In particular, blends of a mixture of the aromatic primary diamines 2,4-diethyl-6-methyl-1,3-benzenediamine and 4,6-diethyl-2-methyl-1,3-benzenediamine (sold as Ethacure® 100 or Ethacure® 100-LC) and an aromatic secondary diamine, N,N′-di-sec-butyl-4,4′-methylenebis(benzeneamine) (sold as Unilink® 4200), are known. However, to date, such blends of aromatic primary diamines and aromatic secondary diamines and especially blends of Ethacure® 100 or Ethacure® 100-LC with Unilink® 4200 do not have a high enough transmission and/or a low enough color for use in certain applications. It would be desirable to have blends of aromatic primary diamines and aromatic secondary diamines which have high enough transmission and/or a low enough color in a range suitable for use in various applications.

### SUMMARY OF INVENTION

This invention provides blends of aromatic primary diamines and aromatic secondary diamines having lower color than previously known blends. So far as is known, prior blends have has significant color because there was no way to obtain blends of low color. Also provided are formulation having higher transmission than previously known formulations.

An embodiment of this invention is a blend which comprises (i) at least one aromatic primary diamine, with which has been blended a color-minimizing amount of at least one N,N-dihydrocarbylhydroxylamine, wherein the aromatic primary diamine is in the form of one benzene ring having two primary amino groups on the ring, which amino groups are meta or para relative to each other, and in which each position ortho (adjacent) to a primary amino group bears an alkyl group, and (ii) at least one aromatic secondary diamine having a Gardner color number no more than about 7, wherein said aromatic secondary diamine either is in the form of one benzene ring having two secondary amino groups on the ring, or is in the form of two benzene rings connected by an alkylene bridge and having one secondary amino group on each ring. Optionally, at least one N,N-dihydrocarbylhydroxylamine has been blended with the aromatic secondary diamine.

Another embodiment of this invention is a formulation which is formed from ingredients comprising (i) at least one aromatic primary diamine, with which has been blended a color-minimizing amount of at least one N,N-dihydrocarbylhydroxylamine, wherein the aromatic primary diamine is in the form of one benzene ring having two primary amino groups on the ring, which amino groups are meta or para relative to each other, said benzene ring also having at least two alkyl groups thereon, (ii) at least one aromatic secondary diamine having a Gardner color number no more than about 7, wherein said aromatic secondary diamine either is in the form of one benzene ring having two secondary amino groups on the ring, or is in the form of two benzene rings connected by an alkylene bridge and having one secondary amino group on each ring, with which at least one N,N-dihydrocarbylhydroxylamine optionally has been blended, (iii) at least one polyol and/or at least one polyetheramine, and optionally (iv) at least one N,N-dihydrocarbylhydroxylamine.

Still another embodiment of this invention is a method for producing a polyurethane, polyurea, or polyurea-urethane, which method comprises blending together (i) at least one aromatic primary diamine, with which has been blended a color-minimizing amount of at least one N,N-dihydrocarbylhydroxylamine, wherein the aromatic primary diamine is in the form of one benzene ring having two primary amino groups on the ring, which amino groups are meta or para relative to each other, said benzene ring also having at least two alkyl groups thereon, (ii) at least one aromatic secondary diamine having a Gardner color number no more than about 7, wherein said aromatic secondary diamine either is in the form of one benzene ring having two secondary amino groups on the ring, or is in the form of two benzene rings connected by an alkylene bridge and having one secondary amino group on each ring, with which at least one N,N-dihydrocarbylhydroxylamine optionally has been blended, (iii) at least one polyol and/or at least one polyetheramine, (iv) optionally at least one N,N-dihydrocarbylhydroxylamine, and (v) at least one isocyanate.

These and other embodiments and features of this invention will be still further apparent from the ensuing description and appended claims.

### FURTHER DETAILED DESCRIPTION OF THE INVENTION

Throughout this document, the term “color minimizing amount” means a quantity sufficient to reduce the existing coloration of a color-possessing aromatic secondary diamine by a measurable amount, provided the resultant reduced coloration is by measurement less than the coloration produced by adding to another sample of the same color-possessing aromatic secondary diamine an equal amount of a clear, colorless inert organic diluent soluble in such diamine.

Those of skill in the art will recognize that there are several ways to name the aromatic secondary diamines in this invention. For example, the structure

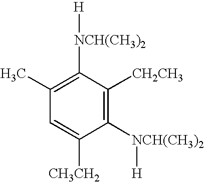

can be called N,N′-di-isopropyl-2,4-diethyl-6-methyl-1,3-benzenediamine, N,N′-di-isopropyl-2,4-diethyl-6-methyl-1,3-phenylenediamine, N,N′-di-isopropyl-3,5-diethyl-2,4-diaminotoluene, or N,N′-di-isopropyl-3,5-diethyl-toluene-2,4-diamine. Similarly, the structure

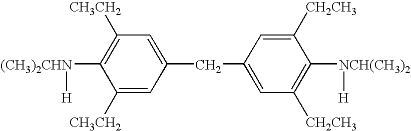

can be called N,N′-di-isopropyl-4,4′-methylenebis(2,6-diethylbenzeneamine), N,N′-di-isopropyl-4,4′-methylenebis(2,6-diethylaniline), or N,N′-di-isopropyl-3,3′,5,5′-tetraethyl-4,4′-diaminodiphenylmethane.

Blends having two or more aromatic primary diamines and/or two or more aromatic secondary diamines are within the scope of this invention. Where an amount is stated to be used or present relative to the aromatic primary diamine or aromatic secondary diamine when two or more such diamines are present, that amount is understood to be relative to the combined total of the aromatic primary diamines or aromatic secondary diamines (e.g., relative to their combined total weight), unless otherwise stated.

In the blends of the invention, the ratio of the aromatic primary diamine to the aromatic secondary diamine are preferably in the range of about 5:1 to about 1:5. More preferably, the ratio is in the range of about 2.5:1 to about 1:2.5. Still more preferred is a ratio of aromatic primary diamine to aromatic secondary diamine in the range of about 1.2:1 to about 1:1.2.

Gardner color is a well known standard color measurement for liquids, and is typically applied to liquids having a yellow, reddish, and/or brownish coloration. As is known in the art, the lower the Gardner color, the clearer (less colored) the liquid appears. See in this connection ASTM standards D1544 (visual standard) and D6166 (instrumentation standard).

Blends of the invention preferably have a Gardner color number no more than about 6. Preferably, the blends have a Gardner color number no more than about 5; more preferred blends have a Gardner color number no more than about 4. Even more preferred are blends having a Gardner color number no more than about 3.5.

The aromatic primary diamines that are part of the blends of the invention preferably have a Gardner color number of no more than about 2.5, more preferably no more than about 2.0. Highly preferred are aromatic primary diamines having a Gardner color number no more than about 1.5.

Aromatic primary diamines in this invention are in the form of one benzene ring having two primary amino groups on the ring, which primary amino groups are meta or para relative to each other, and each position ortho (adjacent) to a primary amino group bears an alkyl group. The alkyl groups ortho to the primary amino groups on the benzene rings may be the same or different. Examples of suitable alkyl groups on the benzene ring of the aromatic primary diamine include methyl, ethyl, propyl, isopropyl, n-butyl, sec-butyl, t-butyl, pentyl, cyclopentyl, hexyl, methylcyclohexyl, heptyl, octyl, cyclooctyl, nonyl, decyl, dodecyl, and the like. Preferred alkyl groups on the benzene rings (ortho to a primary amino group) of the aromatic primary diamines are straight chain or branched chain alkyl groups having from one to about six carbon atoms. Particularly preferred alkyl groups are methyl, ethyl, isopropyl, butyl, and mixtures of two or more of these groups, especially mixtures of methyl groups and ethyl groups. Here, the preference for butyl groups includes n-butyl, sec-butyl, and t-butyl groups. Especially preferred aromatic primary diamines are those in which the two primary amino groups are meta relative to each other. More especially preferred aromatic primary diamines are aromatic primary diamines in which the two primary amino groups are are meta relative to each other and two of the alkyl groups are ethyl groups and the third alkyl group is a methyl group, e.g., 2,4-diethyl-6-methyl-1,3-benzenediamine and 4,6-diethyl-2-methyl-1,3-benzenediamine. Mixtures of 2,4-diethyl-6-methyl-1,3-benzenediamine and 4,6-diethyl-2-methyl-1,3-benzenediamine are very highly preferred. A preferred and commercially available mixture of 2,4-diethyl-6-methyl-1,3-benzenediamine and 4,6-diethyl-2-methyl-1,3-benzenediamine having a N,N′-dihydrocarbylhydroxylamine is Ethacure® 100-LC (a product of Albemarle Corporation), which consist essentially of a major amount by weight (i.e., >50%) of 2,4-diethyl-6-methyl-1,3-benzenediamine and a minor amount by weight of (i.e., <50%) is 4,6-diethyl-2-methyl-1,3-benzenediamine.

The N,N-dihydrocarbylhydroxylamines that are present with the aromatic primary amines in this invention and optionally present with the aromatic secondary diamines in this invention have two hydrocarbyl groups and a hydroxyl group bound to the nitrogen atom. Each hydrocarbyl group is independently a hydrocarbyl group having up to about twenty carbon atoms; preferably, each hydrocarbyl group has up to about 8 carbon atoms. The hydrocarbyl groups of the N,N-dihydrocarbylhydroxylamine may be, for example, alkyl groups (straight chain, branched, or cyclic), alkenyl groups, cycloalkyl groups, cycloalkenyl groups, aryl groups, or aralkyl groups. In the practice of this invention, two or more N,N-dihydrocarbylhydroxylamines can be used.

Also within the scope of this invention is the use of a N,N-dihydrocarbylhydroxylamine in which the two hydrocarbyl groups taken together constitute a single divalent hydrocarbyl group bonded to the nitrogen atom such that the nitrogen atom is part of a heterocyclic ring; such single divalent hydrocarbyl group typically contains up to about 20 carbon atoms and preferably up to about 10 carbon atoms. Since the single divalent hydrocarbyl group has two different carbon atoms singly bonded to the nitrogen atom, such single divalent group may be viewed as two individual hydrocarbyl groups joined together. Thus, throughout this document the term “N,N-dihydrocarbylhydroxylamine” includes such single divalent hydrocarbyl groups that form a heterocyclic ring containing the nitrogen atom as the heteroatom.

Suitable N,N-dihydrocarbylhydroxylamines for the practice of this invention include, but are not limited to, N,N-dipropylhydroxylamine, N,N-diisopropylhydroxylamine, N,N-dibutylhydroxylamine, N,N-diisobutylhydroxylamine, N,N-dipentylhydroxylamine, N,N-dicyclopentylhydroxylamine, N,N-di(2-cyclopentenyl)hydroxylamine, N,N-dihexylhydroxylamine, N,N-diheptylhydroxylamine, N,N-di (methylcyclohexyl)-hydroxylamine, N,N-di(4-methylpentyl)hydroxylamine, N,N-di(dodecyl)hydroxylamine, N,N-di(pentadecyl)hydroxylamine, N,N-di(octadecyl)hydroxylamine, N,N-diphenylhydroxylamine, N,N-di(1-naphthyl)hydroxylamine, N,N-di(2-naphthyl)hydroxyl-amine, N,N-dibenzylhydroxylamine, N,N-di(4-methylbenzyl)hydroxylamine, N,N-bis(2,4-dimethylbenzyl)hydroxylamine, N,N-di(2-phenethyl)hydroxylamine, N,N-di(1-naphthylmethyl)hydroxylamine, and N,N-di(2-naphthylmethyl)hydroxylamine. Considerations when choosing a N,N-dihydrocarbylhydroxylamine include that it not evaporate or decompose during preparation or processing of the blend of which the N,N-dihydrocarbylhydroxylamine is past, and that the N,N-dihydrocarbylhydroxylamine not adversely affect the properties of the blend.

Preferred N,N-dihydrocarbylhydroxylamines are N,N-dialkylhydroxylamines; more preferred are those in which the alkyl groups are straight chain or branched chain alkyl groups, especially those in which each alkyl group, independently, has from one to about six carbon atoms. N,N-diaralkylhydroxylamines are also preferred N,N-dihydrocarbylhydroxylamines, especially those in which each aralkyl group, independently, has from seven to about fourteen carbon atoms. Particularly preferred N,N-dihydrocarbylhydroxylamines in the practice of this invention are N,N-diethylhydroxylamine and N,N-dibenzylhydroxylamine.

The amount of N,N-dihydrocarbylhydroxylaime present with an aromatic primary diamine is typically in the range of about 0.01 wt % to about 1.5 wt % relative to the aromatic primary diamine. Preferred amounts fall in the range of about 0.5 wt % to about 0.75 wt % N,N-dihydrocarbylhydroxylamine relative to the aromatic primary diamine.

The amount of N,N-dihydrocarbylhydroxylamine, when present with an aromatic secondary diamine, is normally present in a color-minimizing amount. Preferably, the N,N-dihydrocarbylhydroxylamine is present in an amount in the range of about 0.01 wt % to about 1.5 wt % relative to the aromatic secondary diamine. More preferably, in the range of about 0.5 wt % to about 0.75 wt % N,N-dihydrocarbylhydroxylamine relative to the aromatic secondary diamine is used. Deviations from these preferred ranges are within the scope of this invention, as amounts other than those in the preferred ranges occasionally may be needed to have a color-minimizing effect. An amount greater than a color-minimizing amount of a N,N-dihydrocarbylhydroxylamine can be present with the aromatic secondary diamine when desired.

The aromatic secondary diamines in this invention are either in the form of one benzene ring having two secondary amino groups on the ring, or are in the form of two benzene rings connected by an alkylene bridge and having one secondary amino group on each ring. The aromatic secondary diamines in this invention have a Gardner color number no more than about 7. Preferably, the aromatic secondary diamines have a Gardner color number no more than about 6; more preferred aromatic secondary diamines have a Gardner color number no more than about 5. Even more preferred are aromatic secondary diamines having a Gardner color number no more than about 4. Aromatic secondary diamines having such low Gardner color values can be produced as described in U.S. patent application [Case K2-7500], file herewith.

Throughout this document, the term “amino hydrocarbyl group” refers to the hydrocarbyl group bound to a nitrogen atom of the aromatic secondary diamine which hydrocarbyl group is not the benzene ring to which the nitrogen atom is bound in order to form the aromatic secondary diamine. The alkylene bridge of the two-benzene-ring secondary diamine has from one to about six carbon atoms; preferably, the alkylene bridge has from one to about three carbon atoms. More preferably, the alkylene bridge has one or two carbon atoms; highly preferred is an alkylene bridge having one carbon atom, i.e., a methylene group.

The amino hydrocarbyl groups of the aromatic secondary diamine generally have from two to about twenty carbon atoms; the amino hydrocarbyl group may be aliphatic (straight chain, branched, or cyclic) or aromatic. Preferably, the amino hydiocarbyl groups are straight chain or branched chain alkyl groups having from three to about six carbon atoms. Examples of suitable amino hydrocarbyl groups include ethyl, propyl, isopropyl, 1-cyclopropylethyl, n-butyl, sec-butyl, cyclobutyl, 2-ethylbutyl, 3,3-dimethyl-2-butyl, 3-pentyl, 2-(4-methylpentyl), 3-penten-2-yl, cyclopentyl, 2,5-dimethylcyclopentyl, 2-cyclopentenyl, hexyl, methylcyclohexyl, menthyl, ionyl, phoryl, isophoryl, heptyl, 2,6,-dimethyl-3-heptyl, cyclooctyl, 5-nonyl, decyl, 10-undecenyl, dodecyl, benzyl, 2,4-dimethylbenzyl, 2-phenylethyl, 1-phenylpentyl, 1-naphthyl, 2-naphthyl, 1-naphthylethyl, and the like. Particularly preferred amino hydrocarbyl groups are isopropyl and sec-butyl.

Aromatic secondary diamines with two secondary amino groups on one benzene ring preferably have the secondary amino groups meta relative to each other. In such preferred aromatic secondary diamines, the amino hydrocarbyl group preferably is a straight chain or branched chain alkyl group having from three to about six carbon atoms.

Preferred aromatic secondary diamines in which one secondary amino group is on each of two benzene rings, where the two benzene rings are connected via an alkylene bridge, have both secondary amino groups para relative to the alkylene bridge. A particularly preferred aromatic secondary diamine is a compound in which the alkylene bridge is a methylene group; this is especially preferred when the amino hydrocarbyl groups are isopropyl or sec-butyl groups.

One type of aromatic secondary diamine in the compositions of the invention is that in which at least one position ortho (immediately adjacent) to each secondary amino group has a hydrogen atom as a substituent. This type of aromatic secondary diamine is preferred; more preferred are aromatic secondary diamines in which both positions ortho to the amino group have hydrogen atoms as substituents. Examples of this type of aromatic secondary diamine include, but are not limited to, N,N′-diisopropyl-1,2-benzenediamine, N,N′-di-sec-butyl-1,3-benzenediamine, N,N′-di(2-butenyl)-1,4-benzenediamine, N,N′-dicyclopentyl-(4-ethyl-1,2-benzenediamine), N,N′-di-sec-butyl-(4-tert-butyl-1,3-benzenediamine), N,N′-di(1-cyclopropylethyl)-2-pentyl-1,4-benzenediamine, N,N′-di(4-hexyl)-(4-methyl-5-heptyl-1,3-benzenediamine), N,N′-dicyclopentyl-4,6-di-n-propyl-1,3-benzenediamine, N,N′-di-sec-butyl-(2,3-diethyl-1,4-benzenediamine), N,N′-di(1-penten-3-yl)-4,5,6-trihexyl-1,3-benzenediamine, N,N′-di(3-hexyl)-2,2′-methylenebis(benzeneamine), N,N′-di(2-cyclopentenyl)-2,3′-methylenebis(benzeneamine), N,N′-diisopropyl-2,4′-methylenebis(benzeneamine), N,N′-di-sec-butyl-3,3′-methylenebis (benzeneamine), N,N-di(3-methyl-2-cyclohexenyl)-3,4′-methylenebis(benzeneamine), N,N′-di(3,3-dimethyl-2-butyl)-4,4′-methylenebis benzene-amine), N,N′-di-sec-butyl-4,4′-methylenebis(benzeneamine), N,N′-di(10-undecenyl)-4,4′-(1,2-ethanediyl)bisbenzeneamine, N,N′-di(phoryl)-3,4′-(1,3-propanediyl)bis(benzeneamine), N,N′-di(2,4-dimethyl-3-pentyl)-2,2′-methylenebis (5-tert-butylbenzeneamine), N,N′-di(2,5-dimethylcyclopentyl)-3,3′-methylenebis (2-methylbenzeneamine), N,N′-di(isophoryl)-3,3′-methylenebis (5-pentylbenzeneamine), N,N′-di(2-hexyl)-3,3′-methylenebis (6-isopropyl-benzeneamine), N,N′-dicyclohexyl-4,4′-methylenebis(3-sec-butylbenzeneamine), N,N′-di(1-cyclopentylethyl)-4,4′-(1,2-ethanediyl)bis(2-methylbenzeneamine), N,N′-diisopropyl-3,3′-methylenebis(2,4-dipentylbenzeneamine), N,N′-di-sec-butyl-3,3′-methylenebis(5,6-diisopropylbenzeneamine), and N,N′-di(menthyl)-4,4′-methylenebis(2,3-di-sec-butylbenzene-amine). An especially preferred aromatic secondary diamine of this type is N,N′-di-sec-butyl-4,4′-methylenebis(benzeneamine).

Another type of aromatic secondary diamine in the compositions of the invention is that in which each position ortho to a secondary amino group (—NHR) bears a hydrocarbyl group. The hydrocarbyl groups ortho to the secondary amino groups on the benzene rings may be the same or different. Examples of suitable hydrocarbyl groups on the benzene ring include methyl, ethyl, propyl, isopropyl, n-butyl, sec-butyl, t-butyl, pentyl, cyclopentyl, hexyl, cyclohexyl, methylcyclohexyl, heptyl, octyl, cyclooctyl, nonyl, decyl, dodecyl, phenyl, benzyl, and the like. When an aromatic secondary diamine of this type is in the form of two benzene rings connected by an alkylene bridge and having one secondary amino group on each ring and the secondary amino group is adjacent (ortho) to the alkylene bridge, the alkylene bridge is considered as a hydrocarbyl group ortho to the secondary amino group. Preferred hydrocarbyl groups on the benzene rings (ortho to a secondary amino group) of the aromatic secondary diamines are straight chain or branched chain alkyl groups having from one to about six carbon atoms; particularly preferred hydrocarbyl groups are methyl, ethyl, isopropyl, butyl, and mixtures of two or more of these groups. Here, the preference for butyl groups includes n-butyl, sec-butyl, and t-butyl groups.

Aromatic secondary diamines in the compositions of this invention having both secondary amino groups on one benzene ring and in which each position ortho (immediately adjacent) to a secondary amino group (—NHR) bears a hydrocarbyl group include, but are not limited to, N,N′-diisopropyl-2,4,6-triethyl-1,3-benzenediamine, N,N′-di-sec-butyl-2,4,6-triethyl-1,3-benzenediamine, N,N′-di-2-pentyl-2,4,6-triethyl-1,3-benzenediamine, N,N′-diisopropyl-(2,4-diethyl-6-methyl-1,3-benzenediamine), N,N′-di-sec-butyl-(2,4-diethyl-6-methyl-1,3-benzenediamine), N,N′-diisopropyl-(4,6-diethyl-2-methyl-1,3-benzenediamine), N,N′-di-sec-butyl-(4,6-diethyl-2-methyl-1,3-benzenediamine), N,N′-di(2-naphthyl)-(4,6-diethyl-2-methyl-1,3-benzenediamine), N,N′-di(2-cyclopentenyl)-(2,4-diisopropyl-6-methyl-1,3-benzenediamine), N,N′-diisopropyl-(2-methyl-4,6-di-sec-butyl-1,3-benzenediamine), N,N′-di-sec-butyl-(2-methyl-4,6-di-sec-butyl-1,3-benzenediamine), N,N′-di(1-cyclopropylethyl)-(2-methyl-4,6-di-sec-butyl-1,3-benzenediamine), N,N′-di(3,3-dimethyl-2-butyl)-(2-ethyl-4-isopropyl-6-methyl-1,3-benzenediamine), N,N′-diisopropyl-2,4,5,6-tetra-n-propyl-1,3-benzenediamine, N,N′-di(3-penten-2-yl)-2,4,5,6-tetra-n-propyl-1,3-benzenediamine, and N,N′-di(4-hexyl)-2,3,5,6-tetraethyl-1,4-benzenediamine. Particularly preferred aromatic diamines having both amino groups on one benzene ring are N,N′-diisopropyl-(2,4-diethyl-6-methyl-1,3-benzenediamine), N,N′-diisopropyl-(4,6-diethyl-2-methyl-1,3-benzenediamine), and mixtures thereof, N,N′-di-sec-butyl-(2,4-diethyl-6-methyl-1,3-benzenediamine), N,N′-di-sec-butyl-(4,6-diethyl-2-methyl-1,3-benzenediamine), and mixtures thereof.

Examples of aromatic secondary diamines of the invention in which one secondary amino group is on each of two benzene rings and in which each position ortho (immediately adjacent) to a secondary amino group (—NHR) bears a hydrocarbyl group include N,N′-diisopropyl-2,2′-methylenebis(6-n-propylbenzeneamine), N,N′-di-sec-butyl-2,2′-methylenebis(3,6-di-n-propylbenzeneamine), N,N′-di(2,4-dimethylbenzyl)-2,2′-methylenebis (5,6-dihexylbenzeneamine), N,N′-diisopropyl-3,3′-methylenebis (2,6-di-n-butylbenzeneamine), N,N′-di(2,4-dimethyl-3-pentyl)-3,3′-methylenebis(2,6-di-n-butylbenzeneamine), N,N′-diisopropyl-4,4′-methylenebis(2,6-diethylbenzeneamine),N,N′-di-sec-butyl-4,4′-methylenebis(2,6-diethylbenzeneamine), N,N′-di(2-hexyl)-4,4′-methylenebis (2,6-diethylbenzeneamine), N,N′-di(1-naphthylethyl)-4,4′-methylenebis(2,6-diisopropylbenzeneamine), N,N′-dicyclobutyl-4,4′-methylenebis(2-isopropyl-6-methylbenzene amine), N,N′-di(1-penten-3-yl)-4,4′-methylenebis(2-methyl-6-tert-butylbenzeneamine), N,N′-di-sec-butyl-4,4′-(1,2-ethanediyl)bis(2,6-diethylbenzeneamine), N,N′-di(1-cyclopentylethyl)-4,4′-(1,2-ethanediyl)bis(2,6-diethylbenzeneamine), N,N′-di(2-ethylbutyl)-4,4′-(1,2-ethanediyl)bis(2,6-diisopropylbenzeneamine), N,N′-di(10-undecenyl)-2,2′-methylenebis(3,4,6-tripentylbenzeneamine), N,N′-di(4-heptyl)-3,3′-methylenebis(2,5,6-trihexylbenzeneamine), N,N′-dimenthyl-4,4′-methylenebis(2,3,6-trimethylbenzeneamine), N,N′-dibenzyl-4,4′-methylenebis(2,3,4,6-tetramethylbenzeneamine), and the like. Particularly preferred aromatic diamines in which one amino group is on each of two benzene rings are N,N′-diisopropyl-4,4′-methylenebis(2,6-diethylbenzeneamine) and N,N′-di-sec-butyl-4,4′-methylenebis(2,6-diethylbenzeneamine).

Optical brighteners are a preferred type of additive to include in the blends of the invention. It has been found that the use of optical brighteners, especially those that can function as blue dyes, minimize the color of aromatic secondary diamines, either used alone or in combination with a N,N-dihydrocarbylhydroxylamine. Two or more optical brighteners can be present in the compositions of this invention. Suitable optical brighteners include, but are not limited to, a mixture of Solvent Violet 13 and Solvent Green 3 (Exalite® Blue 78-13, Exciton Inc., Dayton, Ohio); 2,5-bis(5-t-butyl-2-benzoxazolyl)thiophene (Uvitex OB, Ciba Specialty Chemicals); and 2,2′-(1,2-ethenediyldi-4,1-phenylene)bisbenzoxazole (Uvitex OB ONE, Ciba Specialty Chemicals). A particularly preferred optical brightener in the practice of this invention is Exalite® Blue 78-13.

Generally, the optical brightener is present in a color-minimizing amount. An amount of optical brightener greater than a color-minimizing amount can be used, if desired. The amount of optical brightener is preferably in the range of about 1 ppm to about 100 ppm relative to the aromatic secondary diamine. More preferably, the amount of optical brightener is generally in the range of about 1 wt % to about 5 wt % relative to the aromatic secondary diamine. While the use of larger amounts of optical brighteners is possible, it has been found that there appears to be an upper limit after which further color minimization is not achieved by the addition of more optical brightener, often because the light transmission and/or clarity of the composition, when used in certain applications, becomes too low.

A convenient way to include an optical brightener in a blend of the invention is by the use of a solution of the optical brightener in a polyol. Typically, the optical brightener is made into a solution in the polyol, which solution is then combined with the other components of the blend. Usually, the optical brightener is in the range of about 0.01 wt % to about 10 wt % in the polyol solution; preferably, the optical brightener is in the range of about 0.03 wt % to about 3 wt % in the polyol solution. The suitability of a particular polyol may depend on the end use of the blend. Preferred polyols that can be used include polyether polyols (e.g., Voranol® polyols, Dow Chemical Co.); linear polycaprolactone polyols (e.g., Tone™ polyols, Dow Chemical Co.); and amine-terminated polyols (e.g., Jeffamine® polyols, Huntsman Chemical).

As mentioned above for the N,N-dihydrocarbylhydroxylamines, considerations when choosing an optical brightener or other additive(s) (below) include that the optical brightener and/or other additive(s) not evaporate or decompose during processing of the blend of which such optical brightener(s) and/or other additive(s) is part, and that the optical brightener(s) and/or other additive(s) not adversely affect the properties of the blend.

Other additives may be present in the blend to impart desired properties to the blend, or to an end product made therefrom. One or more such additives may be made part of a composition of the invention. The additive(s) should be chosen so that the desirable properties of the blend are not adversely affected. Examples of such additives include stabilizers, including heat stabilizers and light stabilizers, ultraviolet absorbers, optical brighteners, antifogging agents, weather-proofing agents, antistatic agents, lubricants, surfactants, antioxidants, viscosity reducing agents, dispersants, release agents, processing aids, nucleating agents, and plasticizers. The additive(s) must be compatible with the aromatic primary diamine and the aromatic secondary diamine, and must not materially interfere with the color-minimizing activity of the N,N-dihydrocarbylhydroxylamine.

It is recommended and preferred that the blends of this invention be kept under an inert atmosphere to minimize the amount of oxygen present with the blend. The inert atmosphere is usually comprised of one or more inert gases, such as, for example, nitrogen, helium, or argon. Alternatively, the blends of this invention can be kept under a vacuum, although this is often not practical. Without wishing to be bound by theory, it is believed that oxygen reacts with a portion of the blend to form at least one N-oxide impurity and/or the degradation product of such N-oxide impurity or impurities, which are thought to be the source of at least a portion of the coloration observed in many aromatic secondary diamines. Further, it is recommended and preferred that any preparation or further processing of the blends of the invention be conducted in the substantial absence of oxygen. Here, the term “substantial absence of oxygen” means that oxygen is generally not present, but adventitious amounts of oxygen (e.g., at parts per million levels), although undesired, may be present. It is to be understood that the presence of such adventitious amounts of oxygen are encompassed by the term “substantial absence of oxygen.”

Blends of the invention can be formed in a process which comprises mixing together (i) at least one aromatic primary diamine, with which has been blended a color-minimizing amount of at least one N,N′-dihydrocarbylhydroxylamine, wherein the aromatic primary diamine is in the form of one benzene ring having two primary amino groups on the ring, which amino groups are meta or para relative to each other, and in which each position ortho to a primary amino group bears an alkyl group, and (ii) at least one aromatic secondary diamine having a Gardner color number no more than about 7, wherein said aromatic secondary diamine either is in the form of one benzene ring having two secondary amino groups on the ring, or is in the form of two benzene rings connected by an alkylene bridge and having one secondary amino group on each ring, with which at least one N,N-dihydrocarbylhydroxylamine optionally has been blended. When a blend of the invention comprises one or more additives in addition to the aromatic primary diamine with N,N-dihydrocarbylhydroxylamine and the aromatic secondary amine, the blend can be formed by mixing together the other additive(s) with the aromatic primary diamine with N,N-dihydrocarbylhydroxylamine and the aromatic secondary amine in the desired proportions.

Formulations of the invention are formed from ingredients comprising (i) at least one aromatic primary diamine, with which has been blended a color-minimizing amount of at least one N,N-dihydrocarbylhydroxylamine, wherein the aromatic primary diamine is in the form of one benzene ring having two primary amino groups on the ring, which amino groups are meta or para relative to each other, and in which each position ortho (adjacent) to a primary amino group bears an alkyl group, (ii) at least one aromatic secondary diamine having a Gardner color number no more than about 7, wherein said aromatic secondary diamine either is in the form of one benzene ring having two secondary amino groups on the ring, or is in the form of two benzene rings connected by an alkylene bridge and having one secondary amino group on each ring, with which at least one N,N-dihydrocarbylhydroxylamine optionally has been blended, (iii) at least one polyol and/or at least one polyetheramine, and optionally (iv) at least one N,N-dihydrocarbylhydroxylamine. Optionally, the formulations of the invention further comprise at least one isocyanate. As is well known in the art, other components may also be included in the formulations, such as one or more flame retardants, thermal stabilizers, viscosity reducing agents, and/or surfactants.

The formulations of the invention typically have a light transmission of at least about 20%. Preferred formulations of the invention have a light transmission in the range of about 20% to about 40%. Higher light transmission values (greater amounts of light transmitted) are preferred because such formulations can be used for applications where translucence or transparency and/or clarity are needed or desired.

Blends of the invention can be used as chain extenders in the preparation of polyurethane, polyurea, or polyurethane-urea polymers, in place of the chain extenders that have previously been used in such processes, or the blends can be used in conjunction with one or more known chain extenders, e.g., aromatic primary diamines; the aromatic polyamines of U.S. Pat. Nos. 3,428,610, 4,218,543, 4,595,742, and 4,631,298; polyhydroxyalkanes containing 2-6 carbons and 2-3 hydroxyl groups, such as ethylene glycol, the 1,2- and 1,3-propylene glycols, 1,4-, 1,2-, and 2,3-butanediols, 1,5-pentanediol, neopentyl glycol, 1,6-hexanediol, glycerol, 1,2,4-butanetriol, 1,2,6-hexanetriol; and mixtures of any two or more of the foregoing. Thus, the chain extender or mixture of chain extenders is reacted with an organic polyisocyanate and an active hydrogen-containing organic compound or with a prepolymer thereof having a free —NCO content of at least about 0.1% by weight to form the desired polymer. Whether the blends of the invention are used in place of or instead in conjunction with other chain extenders depends in part on the desired physical properties of the end product. Examples of isocyanates and active hydrogen-containing organic compounds that can be used are taught in, for example, U.S. Pat. No. 4,595,742.

Blends of this invention are to be used as curing agents for epoxy resins, can be used in place of the curing agents that have previously been used to cure such resins, or the blends can be used in conjunction with one or more known curing agents, e.g., aromatic polyamines and/or polyhydroxyalkanes. Whether the aromatic secondary diamines of the invention are used in place of or instead in conjunction with other curing agents depends in part on the desired physical properties of the end product. The epoxy resin may be any epoxy resin, i.e., it may be saturated or unsaturated, aliphatic, cycloaliphatic, aromatic, or heterocyclic. Examples of such resins are taught in Lee et al., Handbook of Epoxy Resins, McGraw-Hill (New York), 1967.

In the methods of the invention, the polyurethane, polyurea, or polyurea-urethane is produced by blending together (i) at least one aromatic primary diamine, with which has been blended a color-minimizing amount of at least one N,N-dihydrocarbylhydroxylamine, wherein the aromatic primary diamine is in the form of one benzene ring having two primary amino groups on the ring, which amino groups are meta or para relative to each other, and in which each position ortho to a primary amino group bears an alkyl group, (ii) at least one aromatic secondary diamine having a Gardner color number no more than about 7, wherein said aromatic secondary diamine either is in the form of one benzene ring having two secondary amino groups on the ring, or is in the form of two benzene rings connected by an alkylene bridge and having one secondary amino group on each ring, with which at least one N,N-dihydrocarbylhydroxylamine optionally has been blended, (iii) at least one polyol and/or at least one polyetheramine, (iv) optionally at least one N,N-dihydrocarbylhydroxylamine, and (v) at least one isocyanate. Usually, the polyol or polyetheramine, aromatic primary diamine with N,N-dihydrocarbylhydroxylamine, aromatic secondary diamine, and when used, optional ingredients, are blended together to form a first mixture, followed by blending this first mixture with the isocyanate to form a second mixture. This second mixture is allowed to cure. The aromatic primary diamine with N,N-dihydrocarbylhydroxylamine and the aromatic secondary diamine may be pre-blended prior to combination with the other ingredients.

The following examples are presented for purposes of illustration, and are not intended to impose limitations on the scope of this invention.

In the Examples below, the Gardner color values were determined instrumentally, using a Color Quest XE spectrophotometer (HunterLab).

### EXAMPLE 1

Diethyl(methyl)-1,3-benzenediamine, as a mixture of its 2,4-diethyl-6-methyl- and 4,6-diethyl-2-methyl-isomers containing ˜2500 ppm of N,N-diethylhydroxyamine (16.5 g; Ethacure® 100-LC, Albemarle Corporation) and N,N′-di-sec-butyl-4,4′-methylenebis(benzeneamine) (Unilink® 4200; 16.5 g) were mixed together under N₂ in a dry box to form a 1:1 (by weight) blend; the blend contained ˜1250 ppm of N,N-diethylhydroxyamine. The composition of this new pale-yellow blend was confirmed by both GC (180° C./5 minutes 10 minutes/270° C.) and GC-MS. After one week under nitrogen in a dry box, the blend remained pale yellow.

### EXAMPLE 2

Example 1 was repeated, using enough material to make 100 grams of the blend. The Gardner color of the blend was 2.7.

### EXAMPLE 3

Several blends of the invention were prepared. The aromatic primary diamine was a mixture of 2,4-diethyl-6-methyl-1,3-benzenediamine and 4,6-diethyl-2-methyl-1,3-benzenediamine containing ˜2500 ppm of N,N-diethylhydroxylamine (Ethacure® 100-LC); the aromatic secondary diamine was N,N′-di-sec-butyl-4,4′-methylenebis(benzeneamine) (Unilink® 4200). Where used, the N,N-dihydrocarbylhydroxylamine was N,N-diethylhydroxylamine. In some blends, an optical brightener was also present; the optical brightener was a mixture of Solvent Violet 13 (CAS # 81-48-1) and Solvent Green 3 (CAS # 128-80-3) (Exalite® Blue 78-13; Exciton, Inc., Dayton, Ohio), which was used as a 0.05 wt % solution of Exalite® Blue 78-13 in a polycaprolactone polyol (Tone™ 32B8, Dow Chemical Co.). In all of the blends, the weight ratio of Ethacure® 100-LC to Unilink® 4200 was 1:1. The Gardner color numbers for the Ethacure® 100-LC and Unilink® 4200 prior to becoming part of the blend and for the blends were determined. Results are summarized in Table 1. The amount of N,N-diethylhydroxylamine and/or Exalite® Blue 78-13 reported in Table 2 is relative to either Ethacure® 100-LC or Unilink® 4200 (because the same quantity of each amine was used). The amounts of N,N-diethylhydroxylamine reported in Table 1 do not include the amount which is already present in the Ethacure® 100-LC. In Table 1, Et₂NOH is an abbreviation for N,N-diethylhydroxylamine.

TABLE 1

| | Gardner color | | | | |
|---|---|---|---|---|---|
| | Ethacure ® | Unilink ® | | | Optical |
| Run | 100-LC | 4200 | Blend | Et₂NOH | brightener |
| 1 | | | 4.5 | 0 | 0 |
| 2 | | 4.4 | 3.1 | 0 | 0 |
| 3 | | | 3.9 | 4 wt % | 0 |
| 4 | 0.7 | 6.5 | 3.8 | 4 wt % | 0 |
| 5 | 0.7 | ~4.5 | 2.7 | 0.3 wt % | 0 |
| 6 | | | 2.1 | 0 | 13.8 ppm |
| 7 | 1 | 7 | 1.5 | 3 wt % | 13.8 ppm |
| 8 | 0.7-1.0 | 6.5 | 0.9 | 4 wt % | 13.8 ppm |

### EXAMPLE 4

Polyurea formulations containing isocyanate (14.9% NCO, Rubinate® 9480, Huntsman Chemical), Jeffamine® D-2000 and Jeffamine® T-5000 (amine-terminated polyols or polyetheramines, Huntsman Chemical), Ethacure® 100-LC (containing ˜2500 ppm of N,N-diethylhydroxylamine), and N,N′-di-sec-butyl-4,4′-methylenebis(benzeneamine) were prepared; two formulations were prepared without Ethacure® 100-LC, for comparative purposes. All ingredients except the isocyanate were mixed together in a blender for five minutes and then degassed in an oven; amounts of the components in this mixture are listed in Table 4. The mixture was placed in one barrel of a two-barrel syringe; the isocyanate was placed in the other barrel. The syringe contents were blended by pushing them through a static mixer onto a steel plate and cured at room temperature. A 1:1 volume ratio of isocyanate to the mixture resulted from the blending of the syringe contents. Amounts of the components of the mixture that was blended with the isocyanate in each formulation are listed in Table 2. The cured formulations were subjected to testing. Properties of the formulations are summarized in Table 2.

TABLE 2

| Component | Formulation 1 | | Formulation 2 | | Formulation 3 | | Formulation 4 | |
|---|---|---|---|---|---|---|---|---|
| Amine-terminated polyol | 41.28 | wt % | 41.28 | wt % | 58.00 | wt % | 53.40 | wt % |
| (Jeffamine ® D-2000) | | | | | | | | |
| Amine-terminated polyol | 5.19 | wt % | 5.19 | wt % | 5.16 | wt % | 5.27 | wt % |
| (Jeffamine ® T-5000) | | | | | | | | |
| Ethacure ® 100-LC | 0 | | 0 | | 18.42 | wt % | 15.08 | wt % |
| N,N′-di-sec-butyl-4,4′- | 53.53 | wt % | 53.53 | wt % | 18.42 | wt % | 26.25 | wt % |
| methylenebis(benzeneamine) | | | | | | | | |
| Mixing temperature | 54° | C. | 54° | C. | 56° | C. | 65° | C. |
| Gel time (cure rate) | 340 | sec. | 340 | sec. | 8 | sec. | 10 | sec. |
| Shore D hardness, 0 sec. | 28 | | 30 | | 37 | | 38 | |
| Shore D hardness, 10 sec. | 21 | | 20 | | 34 | | 36 | |
| Tensile strength | 1300 | psi | 1250 | psi | 2030 | psi | 2360 | psi |
| Elongation | 460% | | 440% | | 340% | | 400% | |
| Modulus (100%) | 540 | psi | 490 | psi | 1110 | psi | 1140 | psi |
| Modulus (300%) | 940 | psi | 890 | psi | 1865 | psi | 1950 | psi |
| Tear strength | 240 | pli | 250 | pli | 411 | pli | 430 | pli |

It is to be understood that the reactants and components referred to by chemical name or formula anywhere in this document, whether referred to in the singular or plural, are identified as they exist prior to coming into contact with another substance referred to by chemical name or chemical type (e.g., another reactant, a solvent, or etc.). It matters not what preliminary chemical changes, transformations and/or reactions, if any, take place in the resulting mixture or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical operation or reaction or in forming a mixture to be used in conducting a desired operation or reaction. Also, even though an embodiment may refer to substances, components and/or ingredients in the present tense (“is comprised of”, “comprises”, “is”, etc.), the reference is to the substance, component or ingredient as it existed at the time just before it was first contacted, blended or mixed with one or more other substances, components and/or ingredients in accordance with the present disclosure.

Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein.

This invention is susceptible to considerable variation within the spirit and scope of the appended claims.

1. A blend which comprises
(i) at least one aromatic primary diamine, with which has been blended a color-minimizing amount of at least one N,N-dihydrocarbylhydroxylamine, wherein the aromatic primary diamine is in the form of one benzene ring having two primary amino groups on the ring, which amino groups are meta or para relative to each other, and in which each position ortho to a primary amino group bears an alkyl group, and
(ii) at least one aromatic secondary diamine having a Gardner color number no more than about 7, wherein said aromatic secondary diamine either is in the form of one benzene ring having two secondary amino groups on the ring, or is in the form of two benzene rings connected by an alkylene bridge and having one secondary amino group on each ring, with which at least one N,N-dihydrocarbylhydroxylamine optionally has been blended.

2. A blend as in claim 1 wherein
said primary diamine of (i) consists essentially of at least one diethyl(methyl)benzenediamine; and/or
said N,N-dihydrocarbylhydroxylamine of (i) is N,N-diethylhydroxylamine.

3. (canceled)

4. A blend as in claim 1 wherein said aromatic secondary diamine of (ii) is either
A) in the form of one benzene ring having two secondary amino groups on the ring, which amino groups are meta or para relative to each other, or
B) in the form of two benzene rings connected by an alkylene bridge and having one secondary amino group on each ring, wherein said alkylene bridge has from one to about three carbon atoms, and wherein each amino group is meta or para relative to the alkylene bridge.

5. A blend as in claim 1 wherein
said aromatic secondary diamine in (ii) is selected from the group consisting of N,N′-diisopropyl-4,4′-methylenebis(2,6-diethylbenzeneamine), N,N′-di-sec-butyl-4,4′-methylenebis(2,6-diethylbenzeneamine), N,N′-di-sec-butyl-4,4′-methylenebis(benzene-amine), and a mixture of any two or more of the foregoing; and/or
a N,N-dihydrocarbylhydroxylamine is present in (ii), and wherein said N,N-dihydrocarbylhydroxylamine is at least one N,N-dialkylhydroxylamine, at least one N,N-diaraalkylhydroxylamine, or a mixture thereof.

6. (canceled)

7. A blend as in claim 1 wherein said primary diamine of (i) consists essentially of at least one diethyl(methyl)benzenediamine, wherein said N,N-dihydrocarbylhydroxylamine of (i) is N,N-diethylhydroxylamine, wherein a N,N-dihydrocarbylhydroxylamine is present in (ii), wherein said N,N-dihydrocarbylhydroxylamine is selected from the group consisting of N,N-diethylhydroxylamine, N,N-dibenzylhydroxylamine, and a mixture thereof, and wherein said aromatic secondary diamine in (ii) is selected from the group consisting of N,N′-diisopropyl-4,4′-methylenebis(2,6-diethylbenzeneamine), N,N′-di-sec-butyl-4,4′-methylenebis(2,6-diethylbenzeneamine), N,N′-di-sec-butyl-4,4′-methylenebis(benzeneamine), and a mixture of any two or more of the foregoing.

8. A blend as in claim 1 which further comprises at least one optical brightener.

9. A blend as in claim 8 wherein said optical brightener is present in an amount in the range of about 1 ppm to about 10 ppm relative to the aromatic secondary diamine.

10-11. (canceled)

12. A process for forming a blend of claim 1, which process comprises mixing together
(i) at least one aromatic primary diamine, with which has been blended a color-minimizing amount of at least one N,N-dihydrocarbylhydroxylamine, wherein the aromatic primary diamine is in the form of one benzene ring having two primary amino groups on the ring, which amino groups are meta or para relative to each other, and in which each position ortho to a primary amino group bears an alkyl group, and
(ii) at least one aromatic secondary diamine having a Gardner color number no more than about 7, wherein said aromatic secondary diamine either is in the form of one benzene ring having two secondary amino groups on the ring, or is in the form of two benzene rings connected by an alkylene bridge and having one secondary amino group on each ring, with which at least one N,N-dihydrocarbylhydroxylamine optionally has been blended.

13. A process as in claim 12 wherein
said primary diamine of (i) consists essentially of at least one diethyl(methyl)benzenediamine; and/or said N,N-dihydrocarbylhydroxylamine of (i) is N,N-diethylhydroxylamine.

14. (canceled)

15. A process as in claim 12 wherein said primary diamine of (i) consists essentially of at least one diethyl(methyl)benzenediamine, wherein said N,N-dihydrocarbylhydroxylamine of (i) is N,N-diethylhydroxylamine, and wherein (i) has a Gardner color of no more than about 2.5.

16. A process as in claim 12 wherein said aromatic secondary diamine of (ii) is either:
A) in the form of one benzene ring having two secondary amino groups on the ring, which amino groups are meta or para relative to each other, or
B) in the form of two benzene rings connected by an alkylene bridge and having one secondary amino group on each ring, wherein said alkylene bridge has from one to about three carbon atoms, and wherein each amino group is meta or para relative to the alkylene bridge.

17. A process as in claim 12 wherein
said aromatic secondary diamine in (ii) is selected from the group consisting of N,N′-diisopropyl-4,4′-methylenebis(2,6-diethylbenzeneamine), N,N′-di-sec-butyl-4,4′-methylenebis(2,6-diethylbenzeneamine), N,N′-di-sec-butyl-4,4′-methylenebis(benzeneamine), and a mixture of any two or more of the foregoing; and/or
a N,N-dihydrocarbylhydroxylamine is present in (ii), and wherein said N,N-dihydrocarbylhydroxylamine is at least one N,N-dialkylhydroxylamine, at least one N,N-diaraalkylhydroxylamine, or a mixture thereof.

18. (canceled)

19. A process as in claim 12 wherein said primary diamine of (i) consists essentially of at least one diethyl(methyl)benzenediamine, wherein said N,N-dihydrocarbylhydroxylamine of (i) is N,N-diethylhydroxylamine, wherein a N,N-dihydrocarbylhydroxylamine is present in (ii), and wherein said N,N-dihydrocarbylhydroxylamine is selected from the group consisting of N,N-diethylhydroxylamine, N,N-dibenzylhydroxylamine, and a mixture thereof, and wherein said aromatic secondary diamine in (ii) is selected from the group consisting of N,N′-diisopropyl-4,4′-methylenebis(2,6-diethylbenzeneamine), N,N′-di-sec-butyl-4,4′-methylenebis(2,6-diethylbenzeneamine), N,N′-di-sec-butyl-4,4′-methylenebis(benzeneamine), and a mixture of any two or more of the foregoing.

20. A formulation which is formed from ingredients comprising
(i) at least one aromatic primary diamine, with which has been blended a color-minimizing amount of at least one N,N-dihydrocarbylhydroxylamine, wherein the aromatic primary diamine is in the form of one benzene ring having two primary amino groups on the ring, which amino groups are meta or para relative to each other, and in which each position ortho to a primary amino group bears an alkyl group,
(ii) at least one aromatic secondary diamine having a Gardner color number no more than about 7, wherein said aromatic secondary diamine either is in the form of one benzene ring having two secondary amino groups on the ring, or is in the form of two benzene rings connected by an alkylene bridge and having one secondary amino group on each ring, with which at least one N,N-dihydrocarbylhydroxylamine optionally has been blended,
(iii) at least one polyol and/or at least one polyetheramine, and optionally (iv) at least one N,N′-dihydrocarbylhydroxylamine.

21. A formulation as in claim 20 which further comprises at least one isocyanate.

22. A formulation as in claim 20 wherein
said primary diamine of (i) consists essentially of at least one diethyl(methyl)benzenediamine; and/or
said N,N-dihydrocarbylhydroxylamine of (i) is N,N-diethylhydroxylamine.

23. (canceled)

24. A formulation as in claim 20 wherein said aromatic secondary diamine of (ii) is either:
A) in the form of one benzene ring having two secondary amino groups on the ring, which amino groups are meta or para relative to each other, or
B) in the form of two benzene rings connected by an alkylene bridge and having one secondary amino group on each ring, wherein said alkylene bridge has from one to about three carbon atoms, and wherein each amino group is meta or para relative to the alkylene bridge.

25. A formulation as in claim 20 wherein
a N,N-dihydrocarbylhydroxylamine is present in (ii), and wherein said N,N-dihydrocarbylhydroxylamine is at least one N,N-dialkylhydroxylamine, at least one N,N-diaraalkylhydroxylamine, or a mixture thereof; and/or
said aromatic secondary diamine in (ii) is selected from the group consisting of N,N′-diisopropyl-4,4′-methylenebis(2,6-diethylbenzeneamine), N,N′-di-sec-butyl-4,4′-methylenebis(2,6-diethylbenzeneamine), N,N′-di-sec-butyl-4,4′-methylenebis(benzeneamine), and a mixture of any two or more of the foregoing.

26. (canceled)

27. A formulation as in claim 20 wherein said primary diamine of
(i) consists essentially of at least one diethyl(methyl)benzenediamine, wherein said N,N-dihydrocarbylhydroxylamine of (i) is N,N-diethylhydroxylamine, wherein a N,N-dihydrocarbylhydroxylamine is present in (ii), wherein said N,N-dihydrocarbylhydroxylamine is selected from the group consisting of N,N-diethylhydroxylamine, N,N-dibenzylhydroxylamine, and a mixture thereof, and wherein said aromatic secondary diamine in (ii) is selected from the group consisting of N,N′-diisopropyl-4,4′-methylenebis(2,6-diethylbenzeneamine), N,N′-di-sec-butyl-4,4′-methylenebis(2,6-diethylbenzeneamine), N,N′-di-sec-butyl-4,4′-methylenebis(benzeneamine), and a mixture of any two or more of the foregoing.

28. A formulation as in claim 20 which further comprises at least one optical brightener.

29. A formulation as in claim 28 wherein said optical brightener is present in an amount in the range of about 1 ppm to about 10 ppm relative to the aromatic secondary diamine.

30-31. (canceled)

32. A method for producing a polyurethane, polyurea, or polyurea-urethane, which method comprises blending together
(i) at least one aromatic primary diamine, with which has been blended a color-minimizing amount of at least one N,N-dihydrocarbylhydroxylamine, wherein the aromatic primary diamine is in the form of one benzene ring having two primary amino groups on the ring, which amino groups are meta or para relative to each other, and in which each position ortho to a primary amino group bears an alkyl group,
(ii) at least one aromatic secondary diamine having a Gardner color number no more than about 7, wherein said aromatic secondary diamine either is in the form of one benzene ring having two secondary amino groups on the ring, or is in the form of two benzene rings connected by an alkylene bridge and having one secondary amino group on each ring, with which at least one N,N-dihydrocarbylhydroxylamine optionally has been blended,
(iii) at least one polyol and/or at least one polyetheramine, (iv) optionally at least one N,N-dihydrocarbylhydroxylamine, and (v) at least one isocyanate.

33. A method as in claim 32 wherein (i) and (ii) are pre-blended.

34. A method as in claim 32 wherein
said primary diamine of (i) consists essentially of at least one diethyl(methyl)benzenediamine; and/or
said N,N-dihydrocarbylhydroxylamine of (i) is N,N-diethylhydroxylamine.

35. (canceled)

36. A method as in claim 32 wherein said primary diamine of (i) consists essentially of at least one diethyl(methyl)benzenediamine, wherein said N,N-dihydrocarbylhydroxylamine of (i) is N,N-diethylhydroxylamine, and wherein (i) has a Gardner color no more than about 2.5.

37. A method as in claim 32 wherein said aromatic secondary diamine is either:
A) in the form of one benzene ring having two secondary amino groups on the ring, which amino groups are meta or para relative to each other, or
B) in the form of two benzene rings connected by an alkylene bridge and having one secondary amino group on each ring, wherein said alkylene bridge has from one to about three carbon atoms, and wherein each amino group is meta or para relative to the alkylene bridge.

38. A method as in claim 32 wherein
a N,N-dihydrocarbylhydroxylamine is present in (ii), and wherein said N,N-dihydrocarbylhydroxylamine is at least one N,N-dialkylhydroxylamine, at least one N,N-diaraalkylhydroxylamine, or a mixture thereof; and/or
said aromatic secondary diamine in (ii) is selected from the group consisting of N,N′-diisopropyl-4,4′-methylenebis(2,6-diethylbenzeneamine), N,N′-di-sec-butyl-4,4′-methylenebis(2,6-diethylbenzeneamine), N,N′-di-sec-butyl-4,4′-methylenebis(benzeneamine), and a mixture of any two or more of the foregoing.

39. (canceled)

40. A method as in claim 32 wherein said primary diamine of (i) consists essentially of at least one diethyl(methyl)benzenediamine, wherein said N,N-dihydrocarbylhydroxylamine of (i) is N,N-diethylhydroxylamine, wherein a N,N-dihydrocarbylhydroxylamine is present in (ii), wherein said N,N-dihydrocarbylhydroxylamine is selected from the group consisting of N,N-diethylhydroxylamine, N,N-dibenzylhydroxylamine, and a mixture thereof, and wherein said aromatic secondary diamine in (ii) is selected from the group consisting of N,N′-diisopropyl-4,4′-methylenebis(2,6-diethylbenzeneamine), N,N′-di-sec-butyl-4,4′-methylenebis(2,6-diethylbenzeneamine), N,N′-di-sec-butyl-4,4′-methylenebis(benzeneamine), and a mixture of any two or more of the foregoing.
